(19) United States
(12) Patent Application Publication
(10) Pub. No.: US 2023/0001176 A1
(43) Pub. Date: Jan. 5, 2023

(54) CONNECTOR, SYSTEM WITH CONNECTOR AND HOSE LINE, AND METHOD FOR CONNECTING A CONNECTOR TO A HOSE LINE

(71) Applicant: B. Braun Melsungen AG, Melsungen (DE)

(72) Inventors: Christoph Otto, Homberg (DE); Rolof Graeper, Spangenberg (DE); Christof Schlitt, Frielendorf (DE)

(21) Appl. No.: 17/781,948

(22) PCT Filed: Dec. 8, 2020
(86) PCT No.: PCT/EP2020/085006

§ 371 Date: Jun. 2, 2022

(30) Foreign Application Priority Data
Dec. 10, 2019 (DE) 10 2019 219 245.9

(51) Int. Cl. A61M 39/12; B29C 65/48; B29C 65/00

(52) U.S. Cl. CPC A61M 39/12; B29C 65/4895; B29C 66/71; B29C 66/73921

(57) ABSTRACT

A connector for connecting a tube to a connection element. The connector includes a housing, which has a coupling portion. The coupling portion is adapted to be at least substantially fluid-tightly connectable to a complementary connection portion of the connection element. The connector includes a tube connection piece, which is adapted to be connectable on the one hand to a tube and on the other hand to the housing in order to make a fluid connection between the tube and the coupling portion of the housing. The housing includes at least one polyolefin.

### CROSS-REFERENCES TO RELATED APPLICATIONS

This application is the United States national phase entry of International Application No. PCT/EP2020/085006, filed Dec. 8, 2020, and claims priority to German Application No. 10 2019 219 245.9, filed Dec. 10, 2019. The contents of International Application No. PCT/EP2020/085006 and German Application No. 10 2019 219 245.9 are incorporated by reference herein in their entireties.

### FIELD

The invention relates to a connector for connecting a tube to a connection element, a system comprising such a connector and a tube, and a method for connecting a connector to a tube.

### BACKGROUND

Administering infusions is a common method of administering medications, vitamins, nutrients, etc. to a human or an animal.

To administer an infusion, routinely, a tube is regularly connected to another tube or to another device such as a valve. Furthermore, one end of a tube is usually connected to a venous catheter. The connections are often so-called Luer connections. Such connections are not only common in infusion technology, but also in other fields of medicine (e.g. transfusion technology, dialysis technology, etc.). Accordingly, the present invention is not limited to applications in infusion technology, but also relates to other technical fields.

The structure of the components of a Luer connection is standardized in the relevant standards, for example ISO 80369-7. The principle of the Luer connection is based on the fact that a Luer external cone (“male”) is inserted into a Luer internal cone (“female”). In the case of a “Luer-slip” type connection, the outer cone is held in the inner cone by the pressing contact. In the case of a connection of the “Luer-Lock” type, in addition a threaded structure with a standardized structure is provided, through which the components of the connection are screwed together.

In addition to Luer connections, other connection geometries are commercially available and standardized in corresponding standards. These are, for example, plug-in connections or screw connections in which cylindrical connecting structures are inserted into each other instead of male and female cones.

It is important that a Luer connection or a connection with a different geometry can be smoothly closed before its use in the course of an infusion, a transfusion, or another medical activity and that it is reliably tight during this use. In addition, it may be desirable that it can be easily released during or after such use.

If a tube is to be connected through such a connection to another element of the infusion system, which is hereinafter referred to as a “connection element”, the tube may be connected to a connector. This connector is intended to be connected to the connection element such that a fluid can flow from the tube into the connection element through the connector.

The geometry of the components of such a connector is based on the one hand on the values specified in the standards and on the other hand on ergonomic aspects. For example, the grip area should be designed in such a way that it may be securely grasped with the hand, especially between the thumb and index finger. Likewise, the components should be as small and light as possible. For example, patients find it annoying when a venous catheter is connected to a large and therefore heavy connector.

Conventional connectors of this type are adhesively bonded to the tube. For this purpose, they are preferably made of a plastic that can be bonded to the tube using organic solvents. This type of bonding is called “solvent bonding”. At least one of the parts to be bonded is treated with an organic solvent and made sticky in a surface area by the solvent. This type of bonding may be implemented quickly and easily by both manual and automatic production. Thus, solvent bonding is a cost-effective bonding method.

Connections using these conventional connectors often experience stresses that increase the torque required to release the connection. Therefore, this torque may only be applied manually with effort or this may not be possible at all. This torque is particularly high if the connector and/or the connection element have been wetted with infusion fluid, disinfectant, or another liquid during connection. Often, tools (for example, pliers) must then be used to loosen the connection, which is time-consuming on the one hand and entails the risk of damage on the other hand. Damage to the infusion set can, for example, lead to uncontrolled leakage of the infusion fluid, which may, inter alia, contain cytotoxic drugs.

In connectors made of polyolefins, significantly lower stresses usually occur, even if they have been wetted with a liquid before being connected. Consequently, connectors made of polyolefins may be easily separated again from the respective connection element. In addition, polyolefins have a high chemical resistance, such that damage such as stress cracking may be reduced or even prevented, even with highly concentrated medications. However, bonding polyolefins to the tube material is difficult. In particular, polyolefins cannot be bonded or not reliably bonded by solvents. Other adhesives, such as acrylate-based adhesives, are required for this purpose, which increases the material costs and the time required to connect the connectors to tubes. This is especially the case when the connectors are manually connected to the tubes. Furthermore, it may be necessary to provide a pin or other protrusion on the connector housing onto which the tube must be pushed for bonding. This is especially the case when the solvent bonding technique is not available. The pin makes the connector relatively large and heavy.

### SUMMARY

An object of the invention is to provide an improved connector for connecting a tube to a connection element, in particular a connector which reduces or avoids the disadvantages described above and provides better comfort for users and/or patients. In particular, it is desirable to provide a connector that may be manufactured in a cost-effective manner and yet is not prone to distortion when connected to a connection element so as to allow easy and safe handling. A further object of the invention is to provide a system comprising such a connector and a tube. A further object of the invention is to provide a method of connecting a connector to a tube.

The connector according to the invention is a connector for connecting a tube to a connection element. The connector comprises a housing having a coupling portion. The coupling portion is adapted to be at least substantially fluid-tightly connectable to a complementary connecting portion of the connection element. The connector further comprises a tube connection piece adapted to be connectable to a tube, on the one hand, and to the housing, on the other hand, to make a fluid connection between the tube and the coupling portion of the housing. The housing comprises at least one polyolefin.

The expression “comprise” is understood here not only in its narrow meaning in the sense of “ to enclose in a spatial manner”, “to surround in a spatial manner” etc., but rather in its general meaning in the sense of “to have”, “to include” etc. This means, for example, that the housing is formed of a polyolefin-containing material or exclusively of polyolefin at least in the area in which it is to come into contact with the connection element, i.e. in particular in the coupling portion, but may also mean that the entire housing material is formed of a polyolefin-containing material or exclusively of polyolefin.

The connection element and the tube are not part of the connector according to the invention per se. However, the connector according to the invention is configured to be connectable to a tube of a predetermined diameter and a connection element of a predetermined connecting geometry, such as a venous catheter.

The expression “connection element” refers to a component which is to be connected to the tube, in particular in order to introduce a fluid from the hose into this component or, conversely, to introduce a fluid from this component into the tube—depending on whether the connector is arranged at the end or at the beginning of the tube, as seen in the direction of flow. The connection element may be a venous catheter, for example. However, the connection element may also be, for example, a coupling element complementary to the connector, which may be connected to a further tube in order to couple the two tubes together.

An advantageous embodiment of the invention comprises attaching a first connector according to the invention to a first tube and attaching a second connector to a second tube. The second connector may also be a connector according to the invention. The first connector and the second connector may be connected to each other in a fluid-tight manner. In this way, the two tubes are connected to each other.

The connector according to the invention comprises a housing with a coupling portion. The coupling portion is adapted to be at least substantially fluid-tightly connectable to a complementary connecting portion of the connection element. That is, the coupling portion has a shape or structure that is complementary to a connecting portion of the connection element. This allows the coupling portion and the connecting portion to be brought into contact or engagement with each other, resulting in an at least substantially fluid-tight connection.

The connector according to the invention is ideally suited as a component of a flexible modular system suitable for connecting different components of an infusion system, a transfusion system, a dialysis system and similar devices. Indeed, a tube connected to the tube connection piece at one end may be connected to different components at the other end. Furthermore, different components (i.e. connection elements) may be connected to the coupling portion of the connector. Furthermore, it is conceivable to market tubes that are already connected to the tube connection piece at one end or at both ends.

The phrase “substantially fluid-tight connection” is used to express that the connection does not leak, or at least does not leak significantly, under the pressure conditions prevailing for the particular application.

An example of the coupling portion of the housing of the connector is a Luer cone according to the design in accordance with the standard ISO 80369-7. Here, the coupling portion of the housing may be a male Luer cone matched to a female Luer cone of the connection element. Conversely, the coupling portion of the housing may also be a female Luer cone that is matched to a male Luer cone of the connection element. In both cases, a threaded structure may be provided by which the housing and the connection element may be screwed (i.e. screw-connected) together to securely retain the male Luer cone in the female Luer cone.

According to the invention, the connector is at least a two-piece connector such that the individual pieces may be manufactured from specifically different materials. This allows the advantageous properties of the different materials to be utilized, while reducing or avoiding the disadvantages associated with the materials.

In particularly preferred embodiments of the invention, the tube connection piece comprises at least one solvent-bondable material.

In this regard, the expression “comprise” with respect to the tube connection piece may mean that it contains or is formed exclusively from at least one solvent-bondable material at least in the region in which it is intended to come into contact with the tube, but may also mean that the entire tube connection piece contains or is formed exclusively from at least one solvent-bondable material.

By using polyolefins for the connector housing, the stresses that lead to an increase in the torque required to release the connection may be reduced or entirely avoided. Thus, a connection between a connector according to the invention and a connection element may be accomplished and released comparatively easily, even if these components have been wetted with a liquid before the connection. Although polyolefin, which is not solvent-bondable, is used for the housing, in embodiments particularly preferred according to the invention, in which the tube connection piece comprises at least one solvent-bondable material, a solvent-bonding technique may be used for the connection to the tube. This may, for example, eliminate the need to apply a separate layer of adhesive to the tube connection piece or to provide a projection on the housing onto which the tube is pushed for bonding. This allows the connector to be relatively small and lightweight, which may be beneficial for user and/or patient comfort. Furthermore, solvent bonding techniques are effective and may be automated, which is economically advantageous for manufacturing the connector according to the invention.

In addition, the use of polyolefins for the connector housing may provide good chemical resistance of this component, reducing the risk of cracking.

The housing is preferably shaped with an ergonomic grip structure. Since the grip area is designed independently of the tube joining area, the grip area may be optimally designed for good handling (i.e. ergonomic). If the connector is not made according to the invention but from a single part, the formation of an ergonomic grip structure may affect the quality of the bonding area. For example, the grip area usually has ridges to allow ergonomic grasping with the hand. There is more material in the area of these ridges than in the areas in between, i.e. the ridges are formed by thickenings in the shell surface. Due to the succession of thicker and thinner areas, sink marks, distortions or other deviations from the nominal geometry can occur inside the housing structure. Such deviations significantly impair the quality of the bond with a hose assembly.

In other words, the invention achieves a decoupling of the material requirements placed on the connector due to the Luer connection or a comparable connection and the material requirements placed on the connector due to the tube joint, such that a particularly well-suited material may be used for each of the two requirements.

The housing and the tube connection piece are preferably manufactured by injection molding or another economically advantageous primary molding process and are connected to each other by pressing, which is also economically advantageous. Overall, the production of the connector according to the invention is thus economical.

The invention does not impose any special requirements on the components which are used together with the connector according to the invention, i.e. the tube and the connection element. Thus, tubes and connection elements already available on the market may be continued to be used.

The material used for the housing contains or consists of polyolefin or a polyolefin mixture. The polyolefin is preferably a polypropylene. The polypropylene used is preferably the material marketed by Borealis AG under the trademark BORMED™ 810MO.

As a solvent-bondable material for the tube connection piece , a plastic is preferably used which is different from the polyolefin used for the housing, wherein the expression “plastic” is understood to mean a material consisting mainly of polymers. The preferred material for the tube connection piece is more preferably a plastic that may be bonded to a conventional tube using methyl ethyl ketone (MEK) or tetrahydrofuran (THF) or a mixture comprising MEK and THF.

It is further preferred that the solvent-bondable polymer is selected from the group consisting of methyl methacrylate-acrylonitrile-butadiene-styrene copolymer (MABS), polystyrene (PS), polyvinyl chloride (PVC), polymethyl methacrylate (PMMA), polycarbonate (PC), styrene-acrylonitrile copolymer (SAN), polyurethane (PU) and styrene-butadiene copolymer (SBC), or such that it comprises one or multiple of the aforementioned polymers. Particularly preferred is methyl methacrylate-acrylonitrile-butadiene-styrene copolymer (MABS), for example the MABS material marketed by the company Ineos Styrolution under the registered trademark TERLUX® 2802.

The tubes used in the medical field (often referred to as “tubing” or “lines”) are infusion tubes for conducting an infusion fluid from an infusion fluid container to a patient, transfusion tubes, dialysis tubes, etc. Conventionally, such hoses are transparent. For example, such tube is made of PVC (polyvinyl chloride) and/or PUR (polyurethane) and/or a styrene-butadiene copolymer (SBC) and/or a polyolefin and/or a silicone. In the case of polyolefin tube, polypropylene is most commonly used.

Although the use of solvent-bondable plastics for the tube connection piece is preferred, the invention is not limited thereto. For example, it is possible for the tube connection piece to be manufactured from a non-solvent-adhesive silicone. Such a tube connection piece would typically be compatible with the use of a silicone tube. The connection between the tube and the tube connection piece may, for example, be made using a UV-activatable silicone-based adhesive. In this way, an advantageous combination of materials results with respect to the tube and the part of the connector that is connected thereto. According to the invention, the housing is manufactured from polyolefin, such that the advantages of polyolefin may also be used in combination with a silicone tube.

The system according to the invention comprises a connector according to the invention and a tube and optionally a connection element. The tube connection piece is preferably connected on the one hand to the tube and on the other hand to the housing, such that there is an at least substantially fluid-tight connection between the tube and the housing. The coupling portion of the housing of the connector is adapted to be connected to the complementary connecting portion of a connection element in an at least substantially fluid-tight manner. The coupling portion of the housing of the connector may also already be connected to the complementary connecting portion of the connection element in an at least substantially fluid-tight manner. The aforementioned components of the system according to the invention may also be present in combination, but separately in a common “kit”.

The method according to the invention is a method for connecting a connector to a tube comprising the steps of:

(A) providing a connector according to the invention with a housing and a tube connection piece,

(B) providing a tube,

(C) connecting the tube to the tube connection piece,

(D) connecting the tube connection piece to the housing.

Steps (C) and (D) can may carried out in any order, but preferably step (C) is carried out first and then step (D).

After carrying out the method according to the invention, a connector is present which is connected to a tube, such that a liquid which is conveyed through the tube may flow through the connector or, conversely, a liquid is introduced into a tube through the connector.

The method may be carried out by first connecting the tube connection piece and the tube and then, in a subsequent step, connecting the tube connection piece to the housing. This procedure may be advantageous for several reasons: Firstly, the tube connection piece may be connected to the tube in advance; when the patient is present, for example in the operating theatre, only the relatively simple step of connecting the tube connection piece to the housing needs to be carried out. Further, the tube connection piece is smaller and lighter than the housing, such that handling during connection is easier if only the tube connection piece and not the entire connector has to be handled. In addition, it may be safely avoided that solvent or other adhesive used for bonding comes into contact with parts of the housing. These advantages of the method are also direct advantages inherent in the connector according to the invention, as these advantages cannot be achieved with conventional connectors.

However, the method may also be carried out in such a way that first the tube connection piece and the housing are connected and in a subsequent step the tube connection piece is connected to the tube.

In both cases, the steps do not have to follow each other immediately. For example, it is conceivable to carry out one step in the factory after manufacturing the components and the other step in the course of use on the patient.

### BRIEF DESCRIPTION OF THE DRAWING FIGURES

Further features, expediencies, and advantages of the invention are described below by means of exemplary embodiments with reference to the attached drawings.

FIG. 1 shows a schematic isometric external view of a connector according to a first embodiment of the present invention.

FIG. 2 shows a schematic cross-sectional view of a connector according to the first embodiment of the present invention.

FIG. 3 shows a schematic cross-sectional view of a connector according to the first example of the present invention, which is additionally connected to a tube and a connection element.

FIG. 4 shows a schematic cross-sectional view of a connector according to a second embodiment of the present invention.

FIG. 5 shows a schematic cross-sectional view of a connector according to a third embodiment of the present invention.

### DETAILED DESCRIPTION

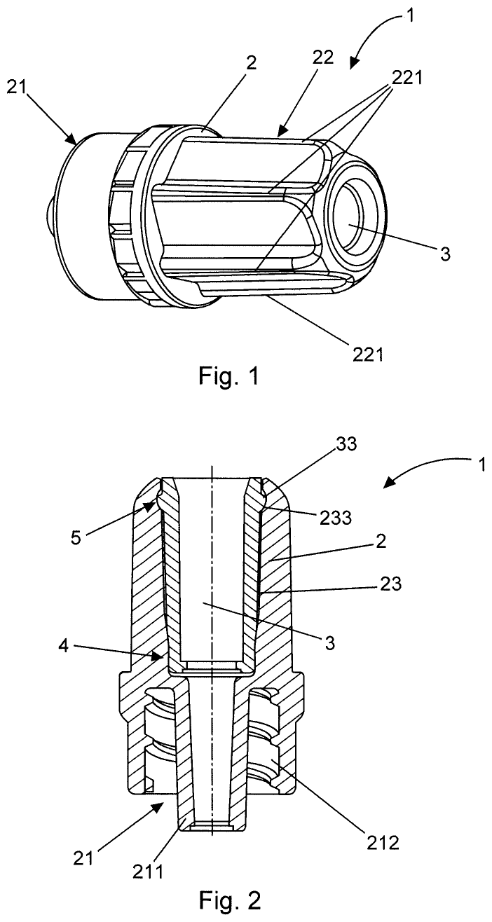

FIG. 1 and FIG. 2 show the connector 1 according to a first embodiment of the present invention in a state in which the connector is connected neither to a tube nor to a connection element.

The connector 1 has a housing 2. The housing 2 is manufactured from a polyolefin. Preferably, the polyolefin used is a polypropylene. In a specific example, a polypropylene marketed by Borealis AG under the trademark BORMED™ 810MO is used. Fillers or excipients may be incorporated into the polyolefin. In alternative embodiments, the housing is only partially made of polyolefin. For example, it may consist of a polyolefin-containing combination material.

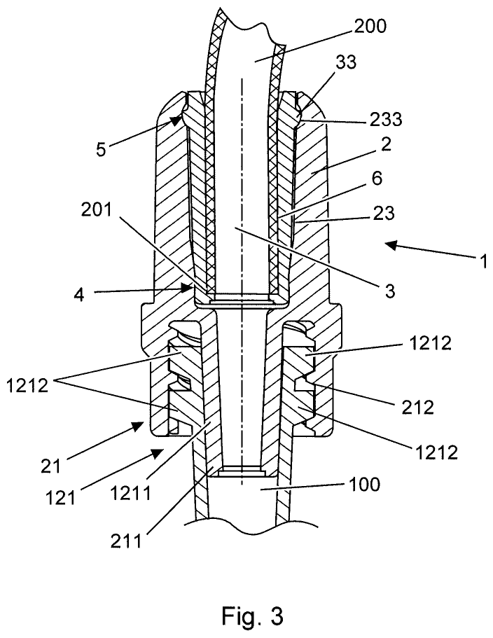

The housing 2 has two ends in the longitudinal direction, which corresponds to the direction of flow of a fluid flowing through the connector 1. At one end, the housing 2 has a coupling portion 21, and at the other end, a recess 23 for receiving a tube connection piece 3, described in detail below. The coupling portion 21 has a shape or structure which is complementary to a connecting portion of the connection element. In the present embodiment, the coupling portion 21 has a male cone 211 that is matched to a female cone of the connection element. In addition, the coupling portion 21 has a threaded structure 212 through which the housing 2 of the connector 1 and the connection element 100 may be screwed together to securely retain the male cone 211 in the female cone. In the present embodiment, the threaded structure 212 is an internally threaded structure. In alternative embodiments, other fastening means may be provided in addition to or as an alternative to the threaded structure, for example a snap-fit fastener. FIG. 3 shows the situation in which a connection element 100 (for example a venous catheter, shown schematically in FIG. 3) is connected to the connector 1 by a connecting structure 121 complementary to the coupling portion 21. The connection element 100 is not part of the connector 1 according to the invention. The connecting structure 121 comprises a female cone 1211 complementary to the male cone 211 of the connector 1. The connecting structure 121 comprises an outer threaded structure 1212 complementary to the inner threaded structure 212 of the connector 1.

For example, the structure of the coupling portion 21 and the complementary connecting portion is the structure of a “Luer-lock” type connection, the structure of which is standardized, for example, in the ISO 80369-7 standard.

Due to a standardized structure of the coupling portion 21, it is advantageously ensured that the connector 1 according to the invention may be used together with commercially available venous catheters, cannulas, spikes for connection to an infusion bag or an infusion bottle, urinary catheters, drains, tube connections, and other medical devices and fittings, which have a connection portion that is also standardized and complementary to the coupling portion 21.

In the present embodiment, the coupling portion 21 has a male cone 211 and an internal thread 212. In alternative embodiments, the connecting portion 21 has a female cone and an external thread, such that a connection with a connection element having a male cone and an internal thread is possible. Preferably, this is again a connection of the “Luer-Lock” type.

The housing 2 has a grip area 22 on its outside. The grip area 22 is shaped in such a way that the connector 1 may be gripped and handled as easily and safely as possible by hand. According to the present embodiment, this is achieved by providing protrusions 221 in a partial area of the outer surface of the housing 2 in the longitudinal direction, between which concave sections lie. In alternative embodiments, instead of such protrusions or in addition thereto, other structural elements may be provided which improve gripping, for example grooves, knobs, roughening, or non-slip overlays.

The connector 1 further comprises a tube connection piece 3. The tube connection piece 3 has a bushing-shaped design, i.e. it has a hollow body. The housing 2 has a recess 23 into which the tube connection piece 3 may be inserted to connect the housing 2 and the tube connection piece 3 together.

The inner wall of the recess 23 and the outer wall of the tube connection piece 3 are adapted to each other. FIG. 2 and FIG. 3 show a situation in which the tube connection piece 3 is located in the recess 23. Here, the outer wall of the tube connection piece 3 seals against the inner wall of the recess 23 in a sealing area 4, such that the connection between the housing 2 and the tube connection piece 3 is at least substantially fluid-tight. The areas of the inner wall of the recess 23 and the outer wall of the tube connection piece 3 that lie in the sealing area 4 are referred to as “sealing portions”. The sealing area 4 may extend over the entire length of the outer wall of the tube connection piece 3 or the inner wall of the recess 23, or a part of this length. The tightness may be achieved by a conical connection between the housing 2 and the tube connection piece 3 in the sealing area 4. However, housing 2 and tube connection piece 3 may also be cylindrical or have a curved shape in sealing area 4. Preferably, the tube connection piece 3 has an outer diameter (diameter of the outer wall of the tube connection piece 3) in the sealing area 4 which is larger than the inner diameter of the recess 23 (diameter of the inner wall of the recess 23) in the sealing area 4. The tube connection piece 3 then has an oversize (difference in diameter) in the sealing area 4 compared to the recess 23. The oversize achieves a particularly tight connection between the tube connection piece 3 and the housing 2. The amount of the oversize is matched to the structure and material of tube connection piece 3 and housing 2. In specific examples, the oversize is 0.1 to 0.25 mm. Such an oversize has turned out to be particularly useful in the case of a housing 2 made of polypropylene and/or a tube connection piece 3 made of methyl methacrylate-acrylonitrile-butadiene-styrene copolymer (MABS).

Furthermore, the housing 2 and the tube connection piece 3 interlock with each other in the form of a connection in a joining area 5. In the present embodiment, this connection is achieved in that a preferably annular recess 233, i.e. a groove, is formed in the region of the end of the housing 2 in the inner wall of the recess 23, and the outer wall of the tube connection piece 3 has a projection 33 corresponding to this recess 233. The projection 33 may run around the entire circumference of the tube connection piece 3 or have interruptions. When the tube connection piece 3 is inserted into the recess 23, the projection 33 engages in the recess 233, since the housing 2 and/or the tube connection piece 3 have at least a certain elasticity. Preferably, the projection 33 has a maximum outer diameter (outer diameter of the projection at its widest location) that is larger than the minimum inner diameter of the recess 23 (inner diameter of the recess 23 at its narrowest location) in the sealing area 4. The projection 33 then has an oversize relative to the recess 233. The oversize additionally achieves a press connection and thus a particularly firm connection overall between the tube connection piece 3 and the housing 2. The size of the oversize is matched to the structure and the material of the tube connection piece 3 and the housing 2 and is preferably selected in such a way that a loose fit is excluded. In specific examples, the oversize is 0.05 to 0.13 mm. Such an oversize has turned out to be particularly useful in the case of a housing 2 made of polypropylene and/or a tube connection piece 3 made of MABS.

In alternative embodiments, the connection may also be provided by the inner wall of the recess 23 having a projection in the joining area 5 and the outer wall of the tube connection piece 3 having a corresponding recess, wherein preferably again there is an oversize. In further alternative embodiments, a sufficiently strong connection between the inner wall of the recess 23 and the outer wall of the tube connection piece 3 is achieved without providing specific recesses and projections. This is preferably achieved by a surface pressure in the sealing area and/or another area of the inner wall of the recess 23 or the outer wall of the tube connection piece 3.

In the present embodiment, the plastic material used as material for the tube connection piece 3 is bondable to a tube using an organic solvent, preferably using MEK or THF or a corresponding mixture. For example, a methyl methacrylate-acrylonitrile-butadiene-styrene copolymer (MABS) may be used. In specific examples, the plastic material marketed by Ineos Styrolution under the registered trademark TERLUX® 2802 is used as material for the tube connection piece 3.

FIG. 3 shows the situation in which a connection element 100 and a tube 200 are connected to the connector 1. Connection element 100 and tube 200 are not components of connector 1.

The tube 200 is connected to the inner wall of the tube connection piece 3 by means of a solvent bonding process, wherein the material on the outside of the hose 200 and the material on the inside of the tube connection piece 3 are bonded together at least in a bonding area 6. In addition, there may be a glue connection between the tube 200 and the tube connection piece.

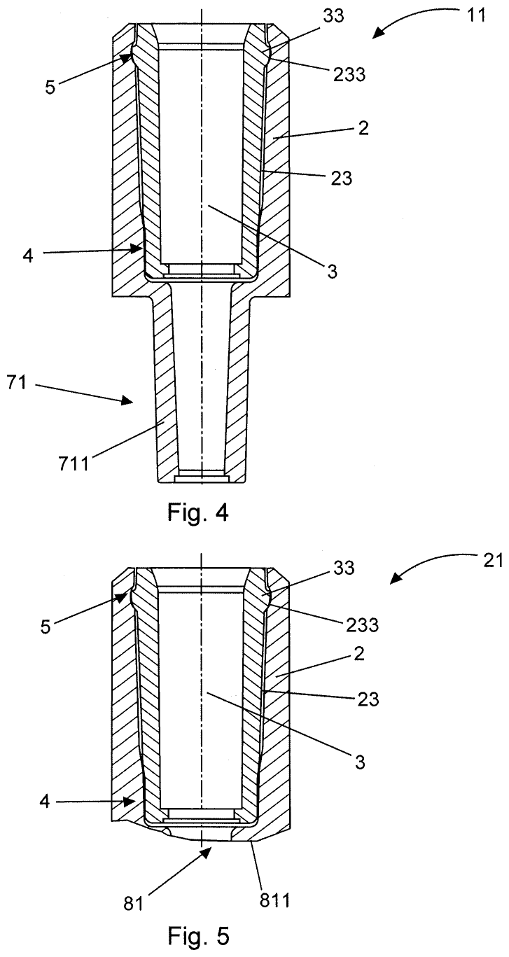

FIG. 4 shows a second embodiment of the connector 11 according to the invention. The second embodiment differs from the first embodiment described above only with regard to the coupling portion 71. The other components of the connector 11 according to the second embodiment correspond to those of the connector 1 according to the first embodiment, such that the same reference signs are used for them. In the second embodiment, the coupling portion 71 has a male cone 711. An internal thread is not present. Preferably, the cone 711 is formed as a Luer cone. When this Luer cone 711 is connected to a corresponding female cone, a “Luer-slip” type connection is present. In alternative embodiments, the coupling portion 71 has a female cone, such that a connection with a connection element having a male cone is possible. Preferably, this is again a connection of the “Luer-slip” type. The advantages of the present invention described above may also be achieved with the connector 11 according to the second embodiment.

FIG. 5 shows a third embodiment of the connector 21 according to the invention. The third embodiment differs from the first embodiment described above only with respect to the coupling portion 81. The other components of the connector 21 according to the third embodiment correspond to those of the connector 1 according to the first embodiment, such that the same reference signs are used for them. In the third embodiment, the connecting portion 81 has an abutment surface 811 which may be connected to a corresponding structure of a corresponding connection element. Optionally, connection elements (not shown in FIG. 5) are provided with which the connection element may be connected to the connector 21. Threads, clamps, sleeves, and the like may be considered as connection elements. The advantages of the present invention described above may also be achieved with the connector 21 according to the third embodiment.

The system according to the invention comprises a connector 1, 11, 21 according to one of the embodiments described above or a variation thereof. Further, the system comprises a tube 200. Optionally, a connection element 100 may be provided. The system comprising a connector 1 according to the first embodiment is shown in FIG. 3. From FIG. 3, it can be seen that the tube connection piece 3 is connected on the one hand to the tube 200 and on the other hand to the housing 2, such that there is an at least substantially fluid-tight connection between the tube 200 and the housing 2. However, a system according to the invention is also present if these connections are not present, but the housing 2, tube connection piece 3 and tube 200 are matched to each other in such a way that these connections may be made. Herein, the connection between tube connection piece 3 and tube 200 is made by solvent bonding.

Furthermore, it can be seen from FIG. 3 that the coupling portion 21 of the housing 2 of the connector 1 is connected to the complementary connecting portion of the connection element 100 in an at least substantially fluid-tight manner. However, the connection element 100 is considered only as an optional component of the system.

In operation, the connector 1, 11, 21 may be connected to a tube 200 using the method according to the invention. For the method according to the invention, the housing 2 and the tube connection piece 3 of the connector as well as a tube 200 are provided. First, according to an embodiment of the method, one end of the hose 200 is treated with an organic solvent. For this purpose, it is wetted with the organic solvent, for example by immersion or a spray. Then, the treated end of the tube 200 is inserted into and brought into contact with the tube connection piece 3 to adhere it thereto. According to an alternative embodiment of the method, the tube connection piece 3 is treated with an organic solvent in an area to be connected to the tube 200. Then, the end of the tube 200 is inserted into the tube connection piece 3 and brought into contact with the treated area of the tube connection piece 3 to bond it thereto. According to another embodiment of the method, both an end of the tube 200 and the tube connection piece 3 are treated with an organic solvent in an area to be connected to the tube 200. Then, the treated end of the tube 200 is inserted into the tube connection piece 3 and brought into contact with the treated area thereof to create an adhesive bond.

In the course of the method according to the invention, the tube connection piece 3 is further connected to the housing 2, wherein this may be done before or after or during making the adhesive connection between the tube connection piece 3 and the tube. Optionally, in the course of the method according to the invention, the connector 1, 11, 21 is connected to the connection element 100, wherein this may be done before or after or during making the adhesive connection between the tube connection piece 3 and the tube.

1. A connector for connecting a tube to a connection element, the connector comprising:
a housing, which has a coupling portion, wherein the coupling portion is adapted to be at least substantially fluid-tightly connectable to a complementary connection portion of the connection element; and
a tube connection piece, which is adapted to be connectable on the one hand to a tube and on the other hand to the housing in order to make a fluid connection between the tube and the coupling portion,
wherein the housing comprises at least one polyolefin.

2. The connector according to claim 1,
wherein the tube connection piece comprises at least one solvent-bondable material.

3. The connector according to claim 1, wherein the housing consists of the polyolefin or a mixture of several polyolefins.

4. The connector according to claim 2, wherein the tube connection piece is made of the solvent-bondable material.

5. The connector according to claim 2, wherein the solvent-bondable material consists of a solvent-bondable plastic or a solvent-bondable mixture of several plastics.

6. The connector according to claim 2, wherein the solvent-bondable material is transparent.

7. The connector according to claim 1, wherein the polyolefin is a polypropylene.

8. The connector according to claim 2, wherein the solvent-bondable material is bondable to the tube using methyl ethyl ketone and/or tetrahydrofuran.

9. The connector according to claim 2, wherein the solvent-bondable material comprises at least one polymer selected from the group consisting of methyl methacrylate-acrylonitrile-butadiene-styrene copolymer, polyvinyl chloride, polymethyl methacrylate, polycarbonate, styrene-acrylonitrile copolymer, polyurethane, and styrene-butadiene copolymer.

10. The connector according to claim 1, wherein the tube to be connected comprises polyvinyl chloride and/or polyurethane and/or styrene-butadiene copolymer and/or polyolefin.

11. The connector according to claim 1, wherein the tube connection piece has a connecting portion via which a form-fitting and/or force-fitting connection can be produced between the tube connection piece and the housing, and
wherein the tube connection piece has a sealing portion by which the connection between the tube connection piece and the housing can be sealed in an at least substantially fluid-tight manner.

12. The connector according to claim 1, wherein the tube connection piece is configured as a bushing intended to receive in its interior an end of the tube, and
wherein the housing has a recess which is intended to receive the bushing in its interior such that there is an at least substantially fluid-tight connection between an outer wall portion of the bushing and an inner wall portion of the recess at least in a sealing region.

13. The connector according to claim 1, wherein the coupling portion is formed as a Luer cone which is intended to form an at least substantially fluid-tight connection with a Luer counter-cone of the connection element.

14. The connector according to claim 13, wherein the Luer cone is an external Luer cone which is adapted to enter into an at least substantially fluid-tight connection with an internal Luer cone.

15. The connector according to claim 1, wherein the tube connection piece is arranged to be connectable to the housing.

16. A system comprising:
a connector according to claim 1; and
a tube,
wherein the tube connection piece is connectable or connected to the tube and to the housing for making an at least substantially fluid-tight connection between the tube and the housing, and
wherein the coupling portion is connectable or connected to the complementary connection portion of a connection element in an at least substantially fluid-tight manner.

17. A method for connecting a connector to a tube comprising the steps of:
(A) providing a connector with a housing and a tube connection piece;
(B) providing a tube;
(C) connecting the tube to the tube connection piece; and
(D) connecting the tube connection piece to the housing,
wherein steps (C) and (D) are performable in any order.

18. The method of claim 17 wherein, wherein step (C) comprises treating at least a portion of the tube connection piece and/or an end of the tube with an organic solvent and bringing the end of the tube into contact with the tube connection piece for solvent bonding.

19. The connector according to claim 10, wherein the tube comprises polyvinyl chloride and/or polyurethane and/or styrene-butadiene copolymer and/or polyolefin.

20. The connector according to claim 15, wherein the tube connection piece is connectable to the housing in a force-fit manner and/or in a form-fit manner.

21. The system according to claim 16, further comprising the connection element.
